(19) United States
(12) Patent Application Publication
(10) Pub. No.: US 2023/0381447 A1
(43) Pub. Date: Nov. 30, 2023

(54) Bubble Machine

(71) Applicants: Joe Hawkes, NEWTOWN, CT (US); Arthur W. Sauer, DANBURY, CT (US)

(72) Inventor: Arthur W. Sauer, DANBURY, CT (US)

(21) Appl. No.: 18/324,723

(22) Filed: May 26, 2023

Related U.S. Application Data
(60) Provisional application No. 63/346,162, filed on May 26, 2022.

(51) Int. Cl. A61M 21/02; A61N 5/06

(52) U.S. Cl. CPC A61M 21/02; A61N 5/0618; A61M 2021/0044

(57) ABSTRACT

A bubble machine designed to illicit a zen and calm environment for the user. The machine includes a square frame mirroring that of a picture frame with a hollow center. Positioned within the hollow center of the frame is an acrylic envelope which is a hollow unit that houses a closed loop. Water is also found within the acrylic envelope. The closed loop is positioned within the acrylic envelope at its outer edge. An air pump is connected to the closed loop suppling a stream of air. The steel loop has a plurality of holes allowing air to escape producing a bubble effect. In addition to the bubbles, a string of LED is positioned outside the acrylic envelope, sitting beneath the envelope, that changes color while in use adding to the calming effect.

### FIELD OF THE INVENTION

The present invention relates generally to relaxation devices. More specifically, the present invention relates to a bubble machine that has air flowing through pipes creating bubbles contained within a pocket that has lights to create a zen experience.

### BACKGROUND OF THE INVENTION

Many people in society suffer from stress due to various factors in their every day life, whether it be from work, school, or just life in general. Stress is a major cause of medical issues and can lead to heart attacks, high blood pressure, depression, and anxiety. Many people turn to medication to help address their stress and anxiety, however, not everyone wishes to take medication and many turn to alternative treatment such as yoga, meditation, and devices that can help provide a calm and soothing environment for them to be in.

The present invention is a device that aims to provide a relaxing and calming environment for someone who suffers from stress. The present invention is compact and moveable, allowing it to be taken on the go to provide a relaxing environment anywhere the user seeks to take the invention.

### SUMMARY OF THE INVENTION

The present invention is a bubble machine with lights. The present invention is designed to have air flowing through a closed stainless-steel loop. The loop has holes along the bottom side of the loop allowing air to be pumped out into the water creating bubbles. Also, along the bottom of the invention is a strand of LED lights that alternate colors. The changing of lights combined with the bubbles produced in the water provide a zen environment for a person needing to manage their stress.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

FIG. 1 is a front view of the present invention.

FIG. 2. is a rear view of the present invention with the backing removed.

FIG. 3. is a rear view of the present invention with the backing in place.

FIG. 4. is a left view of the present invention.

FIG. 5. is top view of the present invention.

### DETAIL DESCRIPTIONS OF THE INVENTION

All illustrations of the drawings are for the purpose of describing selected versions of the present invention and are not intended to limit the scope of the present invention.

Although the invention has been explained in relation to its preferred embodiment, it is to be understood that many other possible modifications and variations can be made without departing from the spirit and scope of the invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

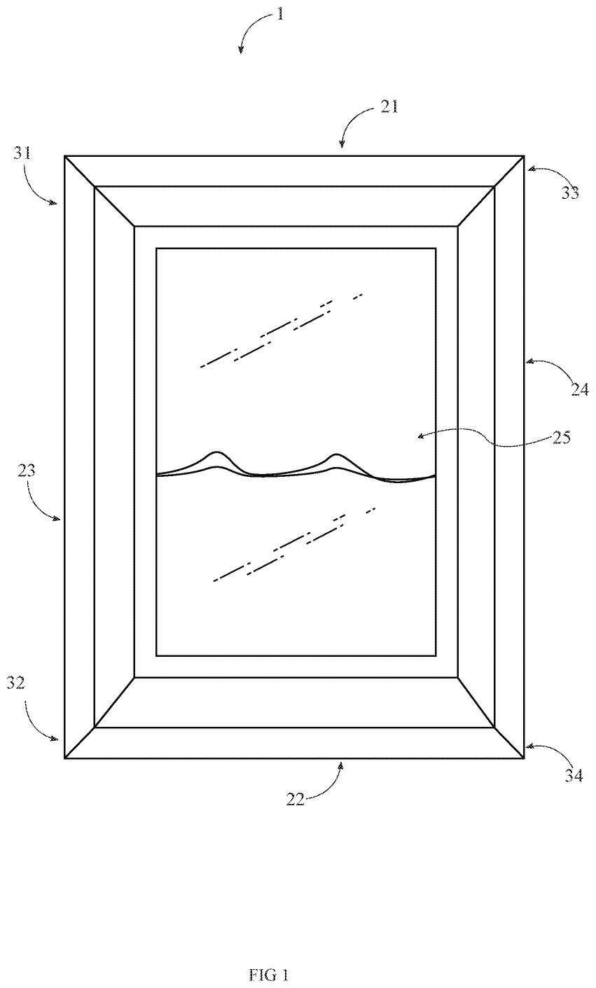

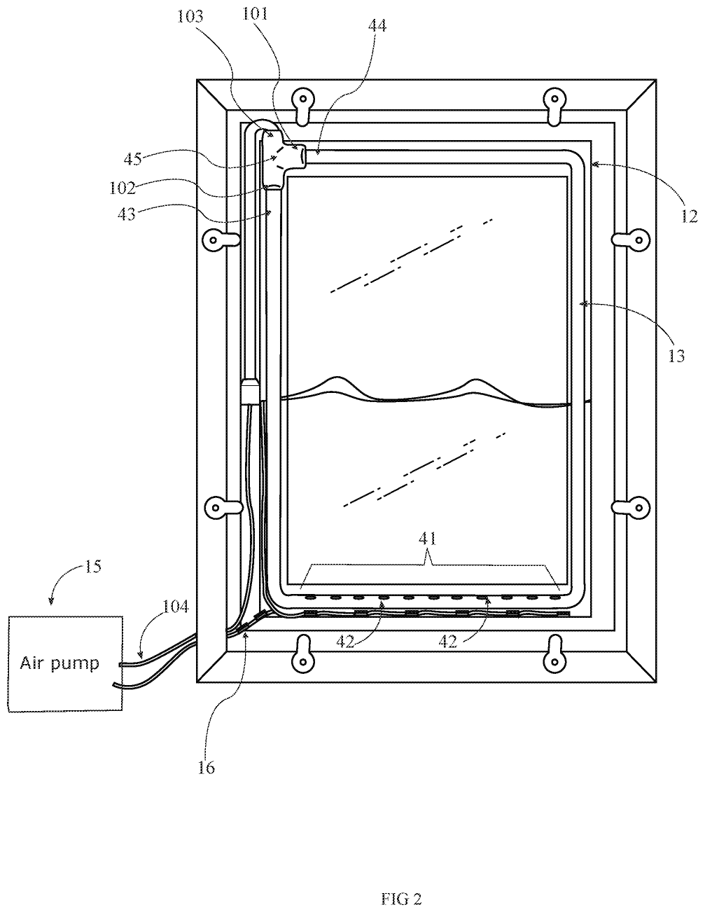

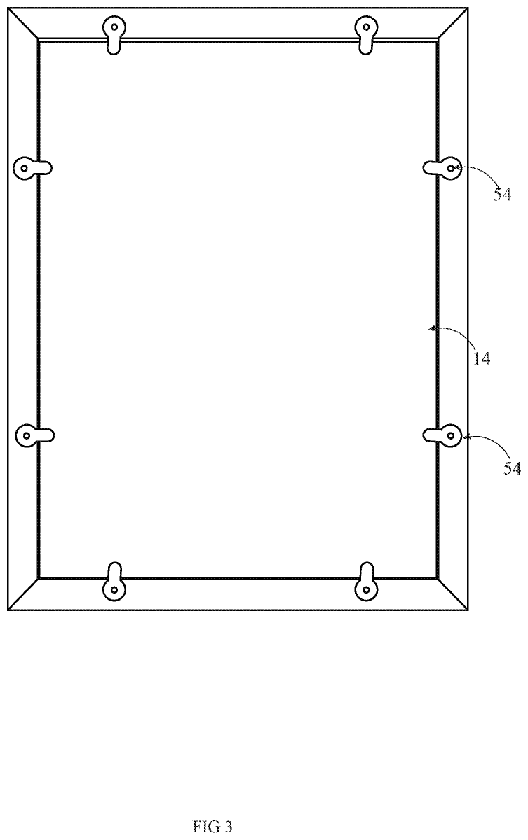

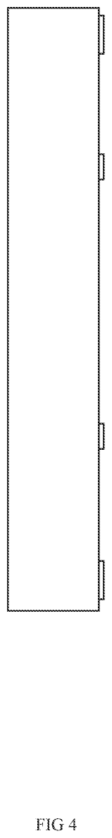

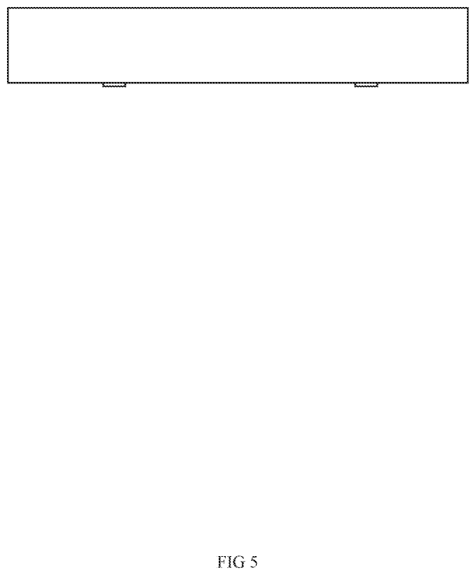

The present invention is a bubble machine. The machine is designed to produce bubbles along with LED lights to produce a soothing and relaxing environment. To do so, as can be seen in FIGS. 1-5, the present invention is a bubble machine. The bubble machine 1 is comprised of a standard frame housing 11 and an acrylic envelope 12. The frame housing 11 is formed by two sets of parallel panels being connected to form a rectangular shaped frame. The frame housing 11 is comprised of a top panel 21, a right panel 24, a bottom panel 22, and a left panel 23. The left panel 23 of the frame housing has a top end 31 and a bottom end 32. The right panel 24 of the frame housing has a top end 33 and a bottom end 34. The top panel 21 of the frame housing is positioned in between the top end 31 of the left panel and the top end of the right panel 33 of the frame housing 11. The bottom panel 22 is positioned in between the bottom end 32 of the left panel and bottom end 34 of the right panel of the frame housing. The frame housing 11 has a hollow center 25. The acrylic envelope 12 sits just behind the frame housing 11. Contained within the acrylic envelope 12, along with water, is a stainless-steel closed loop 13 that has air running continuously through the loop. The closed loop 13 is hollow inside allowing the air a pathway to travel along. The closed loop 13 is perimetrically contained within the acrylic envelope 12. The closed loop 13 has a bottom edge 41 which traverses the bottom panel 22 of the frame housing 11. The closed loop 13 has a plurality of holes 42 that aim towards the top panel 21 of the frame housing 11. This is designed so that when the air travels through the closed loop 13, it is released upwards creating the bubble effect providing the user with the zen experience.

The air is supplied to the loop by use of an air pump 15. The air pump 15 is connected to the frame housing 11 along the lower corner of the right panel. The air pump 15 uses flexible tubing 104 that enters at the lower corner of the frame, at the bottom end 34 of the right panel, travels up the right panel 24 of the frame housing 11 and connects to a copper “T” shaped connector 45. The copper “T” shaped connector 45 is contained in the upper left corner of the acrylic envelope 12. The T-shaped connector 45 has 3 arms, a first arm 101, a second arm 102, and a third arm 103 forming a T-shape. The T-shaped connector 45 is positioned in the upper right corner of the acrylic envelope 12. The other two ends of the “T” each connect to the two ends of the stainless-steel loop, forming a closed circuit. The closed loop has a first inflow end 43 and a second inflow end 44. The first inflow end 43 being connected to a first arm 101 of the T-shaped connector. The second inflow end 44 being connected to a second arm 102 of the T-shaped connector. The air pump 15 being connected to the T-shaped connector 45 by way of flexible tubing 104. The flexible tubing enters through the right panel at the lower corner and traverses up the length of the right panel 104 of the frame housing 11. The flexible tubing 104 connects to the third arm 103 of the T-shaped connector 45.

Sitting just beneath the acrylic envelope 12, along the bottom panel 22 of the frame housing 11, a strand LED lights 16 are positioned underneath the acrylic envelope 12. The LED lights 16 are uniformly spaced and provide changing color lights when the present invention is turned on. The LED lights 16 enter the frame housing 11 on the lower corner along with the air pump 15.

The acrylic envelope 12 is made from clear acrylic plastic and is formed by two acrylic panels being connected with sidewalls forming a hollow housing unit. The acrylic envelope 12 is held in place within the frame housing 11 by backing board 14 that connects to the frame housing 11 by a plurality of clasps 54 to keep the backing board 14 in place. The plurality of clasps 54 are contained along the rear side of the frame housing 11 unit. The backing board 14 is positioned in the hollow center 25 of the frame housing 11 as one might find on a standard picture frame. The backing board 14 is positioned behind the acrylic envelope 12 securing the acrylic envelope 12 in place between the backing board 14 and the frame housing 11. The plurality of clasps 54 are perimetrically positioned along the frame housing 11. Along the bottom edge 41 of the stainless-steel closed loop 13, there are a plurality of holes 42 allowing air to be pumped out of the stainless-steel closed loop 13 when the present invention is turned on creating a soothing bubble pattern in the water. In the preferred embodiment, the air loop is made from stainless steel, however, it can be made by any suitable material that will maintain the functionality of the present invention.

In its preferred embodiment, the current invention provides a soothing environment by turning the air pump 15 on, allowing the air to be pumped into the stainless-steel closed loop 13. With the plurality of holes 42 along the bottom edge of the closed loop 13, air escapes the stainless-steel closed loop 13 out into the water that is contained within the acrylic envelope 12, producing bubbles. When the invention is plugged in, the LED lights 16 turn on and have the ability to change colors, which combined with the bubbles being released from the stainless-steel closed loop 13 produces a relaxing environment for the user of the invention. The present invention can be used on a desk, a nightstand, or can be mounted to the wall.

Although the invention has been explained in relation to its preferred embodiment, it is to be understood that many other possible modifications and variations can be made without departing from the spirit and scope of the invention.

What is claimed is:

1. A bubble machine comprising:
a frame housing;
an acrylic envelope;
a closed loop;
backing board;
an air pump; and
the acrylic envelope being a hollow housing unit.

2. The bubble machine as claimed in claim 1 comprising:
the frame housing having a top panel, a right panel, a bottom panel, a left panel;
the left panel and right panel having a top end and a bottom end;
the top panel of the frame housing being positioned in between the top end of the left panel and the top end of the right panel of the frame housing;
the bottom panel being positioned in between the bottom end of the left panel and bottom end of the right panel of the frame housing; and
the frame housing having a hollow center.

3. The bubble machine as claimed in claim 1 comprising:
the closed loop being perimetrically contained within the acrylic envelope;
the closed loop being hollow inside;
the closed loop having a bottom edge; and
the closed loop having a plurality of holes being positioned along the bottom edge.

4. The bubble machine as claimed in claim 1 comprising:
the closed loop further comprising a T-shaped connector;
the T-shaped connector being defined by 3 arms forming a “T” shape;
the T-shaped connector being positioned in the upper left corner of the acrylic envelope;
the closed loop having a first inflow end and a second inflow end;
the first inflow end being connected to a first arm of the T-shaped connector;
the second inflow end being connected to a second arm of the T-shaped connector; and
the air pump being connected to the T-shaped connector by way of flexible tubing.

5. The bubble machine as claimed in claim 4 comprising:
the flexible tubing entering through the right panel;
the flexible tubing traversing the length of the right panel of the frame housing; and
the flexible tubing being connected to a third arm of the T-shaped connector.

6. The bubble machine as claimed in claim 1 comprising:
a strand of LED lights;
the strand of LED lights entering through the lower right panel of the frame housing; and
the LED lights traversing the bottom panel of the frame housing; and
the strand of LED lights being positioned underneath the acrylic envelope.

7. The bubble machine as claimed in claim 1 comprising:
the backing board being positioned inside the hollow center of the frame housing;
the backing board being positioned behind the acrylic envelope;
the frame housing having a rear side;
a plurality of clasps being perimetrically positioned along the rear side of the frame housing; and
the backing board being attached to the frame housing by way of the plurality of clasps.

8. A bubble machine comprising:
a frame housing;
an acrylic envelope;
a closed loop;
backing board;
an air pump;
the acrylic envelope being a hollow housing unit;
the frame housing having a top panel, a right panel, a bottom panel, a left panel;
the left panel and right panel having a top end and a bottom end; and
the top panel of the frame housing being positioned in between the top end of the left panel and the top end of the right panel of the frame housing.

9. The bubble machine as claimed in claim 8 comprising:
the bottom panel being positioned in between the bottom end of the left panel and bottom end of the right panel of the frame housing; and
the frame housing having a hollow center.

10. The bubble machine as claimed in claim 8 comprising:
the closed loop further comprising a T-shaped connector;
the T-shaped connector being defined by 3 arms forming a “T” shape;
the T-shaped connector being positioned in the upper left corner of the acrylic envelope;
the closed loop having a bottom edge; and
the closed loop having a plurality of holes being positioned along the bottom edge.

11. The bubble machine as claimed in claim 8 comprising:
the air pump being connected to the T-shaped connector by way of flexible tubing;
the flexible tubing entering through the right panel;
the flexible tubing traversing the length of the right panel of the frame housing; and
the flexible tubing being connected to a third arm of the T-shaped connector.

12. The bubble machine as claimed in claim 8 comprising:
the closed loop being perimetrically contained within the acrylic envelope;
the closed loop being hollow inside;
the closed loop having a first inflow end and a second inflow end;
the first inflow end being connected to a first arm of the T-shaped connector; and
the second inflow end being connected to a second arm of the T-shaped connector.

13. The bubble machine as claimed in claim 8 comprising:
a strand of LED lights;
the strand of LED lights entering through the lower right panel of the frame housing; and
the LED lights traversing the bottom panel of the frame housing.

14. The bubble machine as claimed in claim 8 comprising:
the strand of LED lights being positioned underneath the acrylic envelope;
the backing board being positioned inside the hollow center of the frame housing;
the backing board being positioned behind the acrylic envelope;
the frame housing having a rear side;
a plurality of clasps being perimetrically positioned along the rear side of the frame housing; and
the backing board being attached to the frame housing by way of the plurality of clasps.
